(19) United States
(12) Patent Application Publication
(10) Pub. No.: US 2007/0031520 A1
(43) Pub. Date: Feb. 8, 2007

(54) Method of using ajoene as alcohol dehydrogenase inhibitor, composition for removing hangover comprising ajoene and method of preparing the same

(76) Inventors: Sang Hyun Paek, Seoul (KR); Christopher Hansung Ko, Seongnam-si (KR)

Correspondence Address: CANTOR COLBURN, LLP, 55 GRIFFIN ROAD SOUTH, BLOOMFIELD, CT, 06002, US

(21) Appl. No.: 11/498,563

(22) Filed: Aug. 3, 2006

(30) Foreign Application Priority Data
Aug. 3, 2005 (KR) 10-2005-0070869

(51) Int. Cl. A61K 36/8962; A61K 31/11

(52) U.S. Cl. 424/754; 514/702

(57) ABSTRACT

The present invention provides a composition for removing a hangover. In particular, the present invention discloses a method of using ajoene as an alcohol dehydrogenase inhibitor based on the fact that ajoene strongly inhibits alcohol dehydrogenase (ADH) activity. A composition for removing a hangover comprising ajoene suppresses the over-production of acetaldehyde during alcohol metabolism to maintain acetaldehyde concentration in blood at a normal level that can be degraded in the body and eliminated therefrom through normal metabolic and secretory pathways to thereby prevent the hangover after consumption of alcohol, and a method of preparing the same, are also provided. The present invention also provides a novel use of ajoene, which is a natural substance obtained by purification of garlic, and a composition for removing a hangover comprising ajoene, which is harmless to the body and strongly inhibits ADH activity, thereby showing very excellent relieving effect on the hangover.

### CROSS REFERENCE TO RELATED APPLICATIONS

This application claims priority to Korean Patent Application No. 10-2005-0070869, filed on Aug. 3, 2005, and all the benefits accruing therefrom under 35 U.S.C. §119(a), the contents of which are herein incorporated by reference in its entirety.

### FIELD OF THE INVENTION

The present invention relates to a composition for removing a hangover. In particular, the present invention relates to a method of using ajoene as an alcohol dehydrogenase inhibitor based on ajoene having properties that inhibit alcohol dehydrogenase (“ADH”) activity, a composition for removing a hangover comprising ajoene which suppresses the over-production of acetaldehyde during alcohol metabolism to maintain acetaldehyde concentration in blood at a normal level that can be degraded in the body and eliminated therefrom through normal metabolic and secretory pathways to thereby prevent the hangover after consumption of alcohol, and a method of preparing the composition.

### BACKGROUND OF THE INVENTION

Drinking is to ingest ethyl alcohol (referred to herein simply as “alcohol”) into the body. When alcohol is ingested into the body, only 20% of ingested alcohol is absorbed through the stomach and the other 80% is directly absorbed into the small intestine, and then spread to the brain, organs and every cell in the body. Absorbed alcohol is converted to caloric energy by oxidative degradation in the liver through alcohol metabolisim. Alcohol metabolism refers to a series of metabolic cycles including: converting alcohol to acetaldehyde by the action of alcohol dehydrogenase (“ADH”); oxidizing acetaldehyde into acetate by the action of acetaldehyde dehydrogenase (“ALDH”); and finally converting acetate to energy, carbon dioxide and water by enzymatic degradation.

Alcohol, the major biologically active component of a liquor, is degraded into acetaldehyde, which is a known hangover-inducing substance, by alcohol dehydrogenase present in the liver of the body, and acetaldehyde so degraded is further degraded into acetate and water by acetaldehyde dehydrogenase, thereby eliminating them from the body.

The absorption rate of alcohol varies, and relies upon several factors such as the amount of food in the stomach, the kind and amount of liquor, the atmosphere and mood experienced by the consumer when drinking, and the like. People who experience a facial flushing response after some drinking typically have an inherent deficiency in the production of alcohol dehydrogenase and acetaldehyde dehydrogenase that are required for alcohol metabolism, and may therefore have trouble with alcohol consumption. Therefore, such individuals should show restraint in their consumption of alcohol if possible.

Becoming sober often implies that alcohol has been oxidized in the body through alcohol metabolism and is thereby excreted from the body as its metabolites. Acetaldehyde which fails to excrete in this procedure still circulates in the body and stimulates cell therein, which causes a variety of symptoms often called a hangover, with symptoms such as dehydration, tiredness, congestion, headache, nausea, sensitivity to light and noise, trouble sleeping, and the like. It has been reported that while Europeans show high ALDH activity and thus do not frequently experience hangovers, those of Oriental descent show low ALDH activity, which allows excess acetaldehyde levels to be built up in the body upon heavy consumption of alcohol, and which brings about a hangover the next day.

As can be seen from the above process, a hangover may be removed by developing and using a substance that can increase ALDH activity responsible for the degradation of acetaldehyde, inhibit ADH activity responsible for the generation of acetaldehyde to fundamentally maintain a level of acetaldehyde synthesis such that the acetaldehyde produced can be degraded in the body and eliminated therefrom through normal metabolic and secretory pathways without building up in excessive amounts.

However, no successful example of a substance capable of increasing any enzyme's activity by several times has existed up to now. Further, attempts have been made to screen for substances capable of increasing ALDH activity from about 300 or more foods and about 500 or more herbal medicines, but no useful substance has been found therein.

### SUMMARY OF THE INVENTION

Accordingly, the inventors of the present invention have endeavored to devise a novel kind of hangover removing agent through a screening for foods and herb medicines and have found that ajoene, discovered as a component of garlic, strongly inhibits ADH activity. From this finding, the inventors have confirmed that ajoene suppresses the generation of acetaldehyde, a hangover-inducing substance, and therefore the present invention comprises ajoene as an ADH inhibitor.

Thus, in an embodiment, an object of the present invention is to provide a novel use of ajoene being a natural substance isolated from garlic, wherein, in a method, ajoene is used as an alcohol dehydrogenase inhibitor.

Another object of the present invention is to provide a composition comprising ajoene, a natural substance capable of removing a hangover, by suppressing the generation of acetaldehyde, a hangover-inducing substance, wherein the hangover removing properties of the composition are provided by the ajoene.

Still another object of the present invention is to provide a method of preparing the composition, without performing a complicated process, by using a garlic juice that is harmless to the body and environment-friendly.

### BRIEF DESCRIPTION OF DRAWINGS

The above and other objects and features of the instant invention will become apparent from the following description of preferred embodiments taken in conjunction with the accompanying drawings, in which:

FIG. 1 is a schematic procedure showing the generation of ajoene;

FIG. 2 is a schematic procedure showing each step of alcohol metabolism occurred in the body; and

FIG. 3 is a schematic procedure showing each step of purifying ajoene from garlic.

### DETAILED DESCRIPTION OF THE INVENTION

To accomplish the above objects, there is provided a method of using ajoene as an inhibitor for suppressing alcohol dehyrogenase (ADH) activity. This invention will therefore be described below in further detail, with reference to the accompanying drawings.

It will be understood in the following disclosure of the present invention, that as used herein, the term “and/or” includes any and all combinations of one or more of the associated listed items. In addition, the singular forms “a”, “an” and “the” are intended to include the plural forms as well, unless the context clearly indicates otherwise. It will be further understood that the terms “comprise”, “comprises”, and “comprising,” when used in this specification, specify the presence of stated features, integers, steps, operations, elements, components, and/or combination of the foregoing, but do not preclude the presence and/or addition of one or more other features, integers, steps, operations, elements, components, groups, and/or combination of the foregoing. The use of the terms “first”, “second”, and the like, where included, are for purposes of distinguishing elements only, and therefore should not be considered as implying any particular order or sequence unless otherwise specified.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Garlic (Allium sativum L.), used as a raw material from which ajoene is obtained by purifying, is native to Central Asia and is a perennial plant belonging to Allium species of Lilliaceae family. To date, various biologically active substances such as allicin, ajoene, scordinin, fructooligosaccaride, allithiamine, essential oil and so on have been discovered as components of garlic.

Ajoene (isolated as both E or Z isomers of 4,5,9-trithiadodeca-1,6,11-triene-9-oxide) is generated from allicin. As illustrated in FIG. 1, allicin is a sulfur-containing substance generated from allin by the action of allinase, and is degraded even at room temperature into very unstable components such as diallyl disulfide (60%), diallyl sulfide (14%), allyl propyl disulfide (6%) and methyl allyl trisulfide (4-10%) (not shown). When garlic is ground into particles and allowed to remain in the ground state for one day or more, allicin in the ground garlic is converted to ajoene, which comprises two kinds of isomers. Ajoene has been investigated for different properties of biological activity. As discussed by Dr. Eric Block at the University of Albany, State University of New York, USA, ajoene (AJOENE/CPH 14 SO[Please provide the full reference including authors journal, year of publication, volume, and page no.]) as isolated from garlic prevents blood coagulation. According to the Block publication, ajoene can therefore prevent alleviate the blockage of oxygen supply to the brain or heart caused by clogging of a blood vessel due to coagulation of blood in the blood vessel. In this way, various pharmacological effects of garlic including ajoene have been published, however, the using of ajoene as an alcohol dehydrogenase inhibitor has not hitherto been determined.

It has been found that ajoene strongly inhibits alcohol dehydrogenase activity. A method has been devised of using ajoene as a novel inhibitor of alcohol dehydrogenase, an application which was not previously known. Namely, the present invention provides a previously unknown and novel use of ajoene.

Because ajoene is a natural substance isolated from garlic, a widely used food with low toxicity, it is nearly harmless to the body, and therefore has a high margin of safety for its use.

The present invention also provides a composition for removing a hangover, which comprises ajoene as an active ingredient.

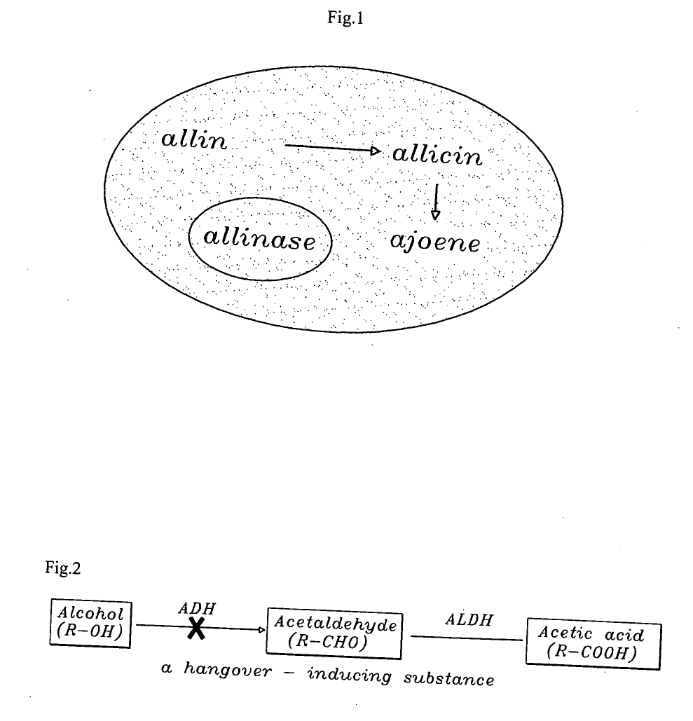

A hangover is a phenomenon that results from consuming (e.g., drinking) alcohol, and is more specifically caused by the oxidation of alcohol through alcohol metabolism in the body and excretion of the alcohol and its metabolic by-products. One metabolite, acetaldehyde, fails to excrete fully and continues to circulate in the body and stimulate cells therein, which causes symptoms such as dehydration, tiredness, congestion, headache, nausea, sensitivity to light and noise, difficulty sleeping, and the like. To prevent such symptoms, and as illustrated in the alcohol metabolism schematic described in FIG. 2, it is useful to delay the breakdown of alcohol into acetaldehyde, by inhibiting ADH activity (as illustrated by a black “X” located on the reaction arrow beneath the term “ADH” in FIG. 2) to maintain the acetaldehyde concentration at a level such that the acetaldehyde is degraded by ALDH in the body without allowing it to accumulate therein, or by improving ALDH activity enough to immediately degrade acetaldehyde into water and acetate and excrete them from the body, and therefore to leave no time for acetaldehyde to accumulate in the body.

As described hereinabove, a substance capable of promoting and enhancing ALDH activity by a factor of several times its base activity has not yet been discovered. Individuals of Oriental descent have a high probability of being genetically unable to produce adequate levels of ALDH. Therefore, to lessen the effects of a hangover in such individuals, it is desirable to regulate the generation of acetaldehyde from alcohol by inhibiting ADH activity to the extent that acetaldehyde can be normally metabolized in the drinker's body without building up to levels that can cause hangover, and thereby be eliminated therefrom.

A composition for removing a hangover according to the present invention, comprises ajoene capable of inhibiting ADH activity. Accordingly, ajoene can prevent the hangover by properly regulating the generation of acetaldehyde through the inhibition of ADH activity. As used herein, “removing” in reference to a hangover means mitigating the severity of, or eliminating entirely, the symptoms of a hangover. Likewise, “prevent” or “preventing” a hangover means mitigating the severity of, or eliminating entirely, the symptoms of a hangover by acting to do so in advance of an action that would induce the hangover (i.e., consumption of alcohol). Thus, a method of using ajoene comprises consuming a composition comprising an amount of ajoene effective to remove a hangover. In an embodiment, the composition is consumed prior to consuming the alcohol.

Preferably, the hangover-removing composition of the present invention contains ajoene in an amount of 1 to 1,000 mg per dose based on 70 kg of body weight.

Here, the content of ajoene included in the composition for removing a hangover is determined based on a dose, desirably administered one or more times daily, for an adult male having a body weight of approximately 70 kg. In an embodiment, the dose is administered once daily. If the content of ajoene per dose of the hangover removing composition is less than 1 mg, the composition does not exhibit sufficient ADH inhibitory activity effective to remove the hangover, whereas if the ajoene content per dose is larger than 1,000 mg, the increased cost associated with excessive use of ajoene is undesirable and can render the composition non-cost effective.

In addition, a composition for removing a hangover comprises a garlic juice. The ajoene is thereby provided as a solution in garlic juice. It is preferable to use the garlic juice prepared by finely grinding raw garlic and filtering it. However, it may be possible to employ as a composition a garlic powder prepared by freeze-drying the garlic juice, in which the garlic powder containing the ajoene may be derived from garlic juice, and is made by a method of extracting ajoene from the garlic juice.

Desirably garlic juice is obtained after the garlic is allowed to age at room temperature in the finely ground state for one day or more. After undergoing such a process or its equivalent, it is possible to obtain a garlic juice containing ajoene, which is generated from allicin after the breakdown of allin into allicin. Further, since volatile substances responsible for the specific garlic aroma and flavor are emitted from the ground garlic and disappear during the aging process, administration of the garlic juice after aging becomes easier and more palatable for the individual consuming the composition.

The garlic juice thus obtained contains ajoene in an amount of about 0.1% to about 10% by weight per 100 g of the garlic juice.

Accordingly, in an embodiment, a composition for removing a hangover of the present invention contains 1 to 10 g of the garlic juice per dose, based on 70 kg of body weight.

Here, the content of the garlic juice included in the composition for removing a hangover is determined based on a dose for the adult male having 70 kg of body weight. If the garlic juice per dose is less than 1 g, the dose does not show sufficient ADH inhibitory activity for removing the hangover, whereas if the garlic juice is greater than 10 g, a possibility exists that the excess garlic juice can cause a change in the composition's taste that may be undesirable when administered orally, and that excess use of garlic juice can increase the cost of production.

In another embodiment, a method of preparing the composition for removing a hangover is characterized by formulating the composition into a tablet or a liquid dosage form.

Here, the composition for removing a hangover is prepared to contain ajoene in an amount of 1 to 100 mg, or to contain the garlic juice in an amount of 1 to 10 g per dose. At this time, the composition for removing a hangover can be formulated into any dosage form known in the art; but it is preferable to formulate the composition into a tablet or a liquid dosage form for the convenience of administration. For example, where the composition for removing a hangover of the present invention is formulated into a tablet form, the composition can be prepared by mixing ajoene, or garlic powder made from drying the garlic juice, with other pharmaceutically acceptable ingredients suitable for preparing a tablet as known in the art, for oral or other administration. Meanwhile, where the composition is formulated into a liquid dose, the composition can be prepared by mixing ajoene or the garlic juice with other soluble and/or miscible and edible beverage ingredients that are known in the art, and which are present in amounts as needed to provide the desired properties for the composition in dose form. Desired properties provided by the added ingredients of the dose forms (e.g., tablet, liquid, etc.) include desired flavor and/or aroma; consistency; dissipation or break-down properties; active ingredient absorption properties; thermal, chemical, or light stabilization of the active ingredient (i.e., the ajoene) or other desired.

Further, in another embodiment, a method of preparing the composition for removing a hangover comprises preparing garlic juice, and mixing the garlic juice with a solution containing other conventional beverage ingredients including beverage ingredients known in the art.

Here, preparing garlic juice is characterized by allowing finely ground garlic or a filtrate obtained by the filtration of the finely ground garlic to age at room temperature for one day or more. Preferably, the filtrate is obtained by finely grinding raw garlic with a wet-grinder, allowing it to age for one day or more and filtering the resultant aged, ground garlic. Alternatively, it may be possible to filter the finely ground garlic in advance, and allow the filtrate obtained therefrom to age for one day or more. Since, through such an aging process, ajoene is generated from allicin which is in turn generated from allin in garlic, garlic juice containing ajoene is obtained. Further, since volatile substances responsible for the garlic aroma and flavor are emitted from the ground garlic disappear during the aging process, the administration of the garlic juice after aging becomes easier and more palatable for the individual ingesting the composition.

It is preferable to mix the garlic juice so prepared with other conventional beverage ingredients known in the art to provide garlic juice in an amount of 1 to 10 g per dose, based on 70 kg of body weight.

Here, it is preferable to employ known beverage ingredients capable of making the composition taste more palatable when consuming (e.g., drinking) the composition and to select beverage ingredients that can harmonize with the flavor of the garlic juice.

The present invention as described above has the following effects.

First, the present invention provides a completely new use of ajoene, namely, a method of using ajoene as an alcohol dehydrogenase inhibitor. Since the method of the present invention employs ajoene which is a natural substance isolated from garlic, and which is nearly harmless to the body and shows very low toxicity and therefore has a high degree of safety, ajoene can be applied to various fields required for the inhibition of alcohol dehydrogenase activity.

Further, the composition for removing a hangover according to the present invention contains a natural substance capable of suppressing the generation of acetaldehyde which is a major cause for the hangover, thereby removing the hangover very effectively. In an embodiment, the natural substance is ajoene. In a more specific embodiment, a method of using ajoene prevents a symptom including dehydration, tiredness, congestion, headache, nausea, sensitivity to light and noise, trouble sleeping, or a combination comprising at least one of the foregoing symptoms. In a specific embodiment, a hangover comprises at least one of the foregoing symptoms.

Furthermore, the method of preparing the composition for removing a hangover according to the present invention is environmentally friendly because it does not require use of any synthetic chemicals to prepare, as the composition can be prepared using garlic juice without employing any complicated processes.

Hereinafter, the present invention will be described in detail with reference to the following examples. These examples are provided to illustrate the present invention, and therefore, should be construed as exemplary and not interpreted as limiting the scope of the present invention.

### EXAMPLE 1

### Purification of Ajoene

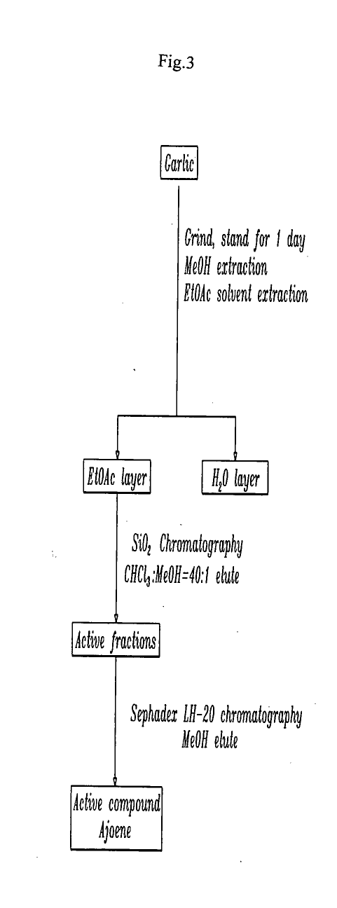

A process for purifying ajoene is described herein, and illustrated as shown in FIG. 3. In the process, garlic (1 kg) was finely ground with a wet-grinder, and allowed to age at room temperature in the ground state for one day. The ground garlic was extracted with methanol (MeOH), concentrated under reduced pressure, the concentrated extracts were dissolved in water and the aqueous solution of extracts was further extracted with ethyl acetate (EtOAc).

The ethyl acetate solvent layer was concentrated and subjected to silica gel TLC (thin layer chromatography) using CHCl₃/MeOH (20:1(v/v)) as the eluting solvent, to obtain the fraction eluting at an R_{f} value of 0.3, and which showed ADH inhibitory activity. In order to purify a large quantity of ajoene based on the above, the EtOAc solvent layer was concentrated and subjected to silica gel column chromatography using CHCl₃/MeOH (40:1(v/v)) as an eluting solvent, and then fractionated. All the fractions having a band (TLC on silica, CHCl₃/MeOH 20:1 v/v)) with an R_{f} value of 0.3 were collected therefrom and concentrated under reduced pressure. Then, the resulting concentrate was subjected to Sephadex LH-20 column chromatography to purify an active ingredient. The fraction showing ADH inhibitory activity was isolated and the purified ajoene was obtained therefrom. The aforementioned purification process is shown schematically in FIG. 3.

Confirmation through UV, IR and NMR analysis showed that ajoene so purified from garlic has the same structure as a known sample of ajoene.

### TEST EXAMPLE 1

### Examination of ADH Inhibitory Activity of Ajoene

ADH inhibitory activity of ajoene obtained in Example 1 was examined by means of a cloned human ADH assay wherein human ADH is treated with ajoene at different concentrations, and the results are shown in Table 1 below. Here, the ADH assay employed 0.1 M glycine buffer (pH 10) as a reaction buffer and 2.4 mM NAD⁺ and 33 mM ethanol in a subtrate. ADH inhibitory activity was determined by measuring the absorbance of NADH at 340 nm using a microplate reader.

TABLE 1

| Concentration of ajoene (μg/ml) | ADH inhibitory activity (%) |
|---|---|
| 0.1 | 0 |
| 0.9 | 50 |
| 1 | 60 |
| 10 | 85 |
| 50 | 100 |
| 100 | 100 |

Table 1 above shows the results of measuring the inhibitory effect of ajoene isolated from garlic on ADH activity. From the results, it can be seen that ajoene shows an inhibitory concentration of 50% (IC₅₀) at a concentration of 0.9 μg/ml, and therefore ajoene inhibits ADH activity very strongly.

### EXAMPLE 2

### Preparation of a Composition for Removing a Hangover Comprising Ajoene

Ajoene as obtained in Example 1 was dissolved in 200 nm samples of orange juice in amounts of 100 mg, 500 mg and 1,000 mg per sample to thereby obtain Compositions 1, 2, and 3 (respectively), each representing a single dose, for removing a hangover.

### TEST EXAMPLE 2

[Please provide the trade name or active ingredient, as well as dosage, for the commercial hangover relieving agent used for comparison] Twenty adult males each of about 70 kg of body weight and who were known to suffer from the after-effects of a previous night's consumption of alcohol were selected. One of the Compositions 1-3 for removing a hangover as prepared in Example 2, or a commercial hangover relieving agent, were administered to each of the subjects before consuming alcohol, whereupon each subsequently consumed alcohol in the form and amount of one bottle of beer and two bottles of soju with mixing. It was estimated whether each subject would have a hangover the next morning based on the following criteria and the average estimated results shown in Table 2 below.

Criteria for estimation of hangover severity: very severe hangover, 5 points,; severe hangover, 4 points; normal hangover, 3 points; slight hangover, 2 points; negligible hangover, 1 point; no hangover, 0 point.

TABLE 2

| | | | Commercial hangover |
|---|---|---|---|
| Composition 1 | Composition 2 | Composition 3 | relieving agent |
| 1.3 | 1.0 | 0.7 | 3.3 |

From the results of Table 2 above, it can be seen that the compositions for removing a hangover according to the present invention all show a significantly high relieving effect on the hangover than the commercial hangover-relieving agent.

### EXAMPLE 3

### Preparation of a Composition for Removing a Hangover Comprising Garlic Juice

Raw garlic was washed, dried and finely ground using a wet-grinder. After the ground garlic was allowed to age at room temperature for about a day and a half, it was filtered to obtain garlic juice. The garlic juice thus prepared contained about 10% by weight of ajoene. The garlic juice was then mixed with 200 ml samples of orange juice in amounts of 1 g, 5 g and 10 g, respectively, to obtain Compositions 4, 5, and 6 (respectively) for removing a hangover.

### TEST EXAMPLE 3

The evaluation procedure used for Compositions 4-6 was the same as that described for Compositions 1-3 in Test Example 2. The day after consuming alcohol, it was determined whether the test subjects using Compositions 4-6 had a hangover according to the same method and criteria as described in Test Example 2. The averaged results for Compositions 4-6 are shown in Table 3 below.

TABLE 3

| | | | Commercial hangover |
|---|---|---|---|
| Composition 4 | Composition 5 | Composition 6 | relieving agent |
| 1.3 | 1.0 | 0.7 | 3.3 |

As can be seen from Table 3 above, Compositions 4-6 each showed little or no difference in the relieving effect on the hangover between the use of ajoene itself as purified from garlic (Example 2) and that of the garlic juice containing ajoene (Example 3). Further, both Examples 2 and 3 performed well in reducing the severity of a hangover, and both significantly out performed the commercial hangover relieving agent.

While the present invention has been shown and described with respect to particular embodiments, it will be apparent to those skilled in the art that many changes and modifications may be made without departing from the spirit and scope of the inventions as defined in the appended claims.

What is claimed is:

1. A method of using ajoene as an inhibitor for suppressing alcohol dehyrogenase (ADH) activity.

2. The method according to claim 1, wherein using ajoene removes a hangover.

3. The method according to claim 1, wherein using ajoene comprises consuming a composition comprising an amount of ajoene effective to remove a hangover, prior to consuming alcohol.

4. The method according to claim 3, wherein the amount of ajoene comprises I to 1,000 mg based on 70 kg of body weight.

5. The method according to claim 1, wherein using ajoene prevents a symptom including dehydration, tiredness, congestion, headache, nausea, sensitivity to light and/or noise, trouble sleeping, or a combination comprising at least one of the foregoing symptoms.

6. A composition for removing a hangover comprising ajoene.

7. The composition according to claim 6, wherein a dose of the composition comprises ajoene in an amount of 1 to 1,000 mg per dose based on 70 kg of body weight.

8. The composition according to claim 7, wherein the dose is formulated into a tablet or a liquid dosage form.

9. The composition according to claim 6 wherein the ajoene is provided as a solution in garlic juice.

10. The composition according to claim 9, wherein the garlic juice is obtained by finely grinding garlic, aging the ground garlic at room temperature for one day or more and filtering the resultant ground garlic.

11. The composition according to claim 9, which comprises the garlic juice in an amount of 1 to 10 g per dose based on 70 kg of body weight.

12. A method of preparing a composition for removing a hangover, comprising formulating a composition comprising ajoene into a tablet or a liquid dosage form.

13. The method according to claim 12, wherein the ajoene is used as a solution in garlic juice when the composition is in liquid dosage form.

14. The method according to claim 12, wherein the ajoene is derived from garlic juice when the composition is in a tablet form.

15. The method of claim 14, wherein the ajoene is used in the composition as a garlic powder.

16. A method of preparing a composition for removing a hangover comprising preparing garlic juice, and mixing the garlic juice with beverage ingredients.

17. The method according to claim 16, wherein preparing garlic juice comprises allowing finely ground garlic or a filtrate obtained by the filtration of finely ground garlic to age at room temperature for one day or more.

18. The method according to claim 17, wherein the garlic juice is mixed with the beverage ingredients in an amount of 1 to 10 g per dose based on 70 kg of body weight.
